(19) United States
(12) Patent Application Publication
(10) Pub. No.: US 2010/0016575 A1
(43) Pub. Date: Jan. 21, 2010

(54) BACTERIAL CELLULOSE-CONTAINING FORMULATIONS LACKING A CARBOXYMETHYL CELLULOSE COMPONENT

(76) Inventors: Zhi-Fa Yang, San Diego, CA (US); Robert Raczkjowski, Chula Vista, CA (US); Larry B. Rubic, San Diego, CA (US); Melvin Johnny Mazyck, San Diego, CA (US); Kathleen M. Deely, Naperville, IL (US)

Correspondence Address: SUTHERLAND ASBILL & BRENNAN LLP, 999 Peachtree Street, NE, Atlanta, GA, 30309-3996, US

(21) Appl. No.: 12/173,592

(22) Filed: Jul. 15, 2008

(51) Int. Cl. C07H 1/08

(52) U.S. Cl. 536/56

(57) ABSTRACT

A method for the production of a bacterial cellulose-containing formulation that lacks a carboxymethyl cellulose component. The method includes providing a bacterial cellulose product, mixing the bacterial cellulose product with a polymeric thickener and/or a precipitation agent, lysing the bacterial cells from the bacterial cellulose product or the mixture of the bacterial cellulose product and the polymeric thickener or precipitation agent, and co-precipitating the resultant mixture with a water-miscible non-aqueous liquid. The resultant bacterial cellulose formulation includes at least one bacterial cellulose material and at least one polymeric thickener. The bacterial cellulose formulation, may be used in food compositions.

### BACKGROUND

1. Field of the Art

The present embodiments relate generally to a novel bacterial cellulose formulation, and more particularly to a bacterial cellulose formulation lacking a carboxymethyl cellulose component, and a method for making the bacterial cellulose formulation.

2. Background of the Art

Bacterial cellulose is a broad category of polysaccharides that exhibit highly desirable properties, even though such compounds are essentially of the same chemical structure as celluloses derived from plant material. As the name purports, however, the source of these polysaccharides are bacterial in nature (produced generally by microorganisms of the Acetobacter genus) as the result of fermentation, purification, and recovery thereof. Such bacterial cellulose compounds are comprised of very fine cellulosic fibers having very unique dimensions and aspect ratios (diameters of from about 40 to 100 nm each and lengths of from 0.1 to 15 microns or longer) in bundle form (with a diameter of 0.1 to 0.2 microns on average). Such an entangled bundle structure forms a reticulated network structure that facilitates swelling when in aqueous solution thereby providing excellent three-dimensional networks. The three-dimensional structures effectuate proper and desirable viscosity modification as well as suspension capabilities through building a yield-stress system within a target liquid as well as excellent bulk viscosity. Such a result thus permits highly effective suspension of materials (such as foodstuffs, as one example) that have a propensity to settle over time out of solution, particularly aqueous solutions. Additionally, such bacterial cellulose formulations aid in preventing settling and separation of quick-preparation liquid foodstuffs (i.e., soups, chocolate drinks, yogurt, juices, dairy, cocoas, and the like), albeit with the need to expend relatively high amounts of energy through mixing or heating to initially reach the desired level of suspension for such foodstuffs.

The resultant fibers (and bundles) are insoluble in water and, with the capabilities noted above, exhibit polyol- and water-thickening properties. One particular type of bacterial cellulose, microfibrillated cellulose, typically is provided in an uncharged state and exhibits the ability to associate without any added influences. However, without any extra additives to effectuate thickening or other type of viscosity modification, the resultant systems will themselves exhibit high degrees of instability, particularly over time periods associated with typical shelf life requirements of foodstuffs. Consequently, certain co-agents, like carboxymethylcellulose (CMC), have been introduced to bacterial cellulose products to provide stabilization and dispersion improvements. Such co-agents may be combined with bacterial cellulose products such as through adsorption to the fibers thereof, followed by spray drying (without any co-precipitation steps), most likely transferring negative charges on the CMC to the bacterial cellulose fibers themselves. Such charges appear to provide repulsion capabilities that prevent the fiber bundles from relaxing the network formed. The selection of a proper CMC has been known to greatly affect the rheological properties of the target bacterial cellulose, most likely due to the salt and acid sensitivities of certain CMC products. For example, see U.S. Patent Application Publication Number 2007/0197779, incorporated herein by reference in its entirety.

Although CMC inclusion has been shown to provide improvements in bacterial cellulose utilization, there are applications for which CMC inclusion is not desired. This may be, at least in part, because CMC is produced by chemical modifications to natural celluloses, and as such is considered an industrial chemical. An example of such an application is the food industry, in which a surge in natural labeling is arising as a global trend. While microfibrous cellulose formulations containing a significant amount of CMC, including those sold by CP Kelco under the trademarks AxCel™ PX and AxCel™ PG, are currently available in the marketplace, these products have limited use in the food industry because of their CMC content. Because CMC has been regarded as an indispensable component for microfibrous cellulose functionality, it has, up until now, been believed that microfibrous cellulose formulations generally would be excluded from certain food applications.

The presence of CMC has also limited the use of microfibrous cellulose formulations, including AxCel™, in certain industrial uses. For example, CMC has been found detrimental in certain cationic compatible systems. CMC is negatively charged and it is believed it will react with positively charged molecules, such as cationic surfactants, proteins, etc. to form a complex that may precipitate out of the solutions. Cationic guar has been used with microfibrous cellulose to overcome limitations in cationic systems; however, it too has been considered an industrial chemical that is unsuitable for food applications.

### BRIEF DESCRIPTION OF THE EMBODIMENTS

In view of the foregoing, a need exists for a CMC free version of microfibrous cellulose formulations that can be used in the food industry.

Accordingly, the embodiments described herein encompass methods for the production of a bacterial cellulose-containing formulation. In one embodiment, an exemplary method includes the following steps: a) providing a bacterial cellulose product; b) optionally lysing the bacterial cells from the bacterial cellulose product; c) mixing the resulting bacterial cellulose product of either step “a” or “b” product with a polymeric thickener selected from the group consisting of at least one charged polymer, at least one precipitation agent, and any combination thereof; and d) co-precipitating the mixture of step “c” with a water-miscible nonaqueous liquid.

The embodiments also encompass a method including the following steps: a) providing a bacterial cellulose product; b) optionally lysing the bacterial cells from the bacterial cellulose product; c) mixing the resulting bacterial cellulose product of either step “a” or step “b” with at least one precipitation agent; and d) co-precipitating the mixture of step “c” with a water-miscible nonaqueous liquid. In this method, the precipitation agent is selected from the group consisting of a xanthan product, pectin, alginates, gellan gum, welan gum, diutan gum, rhamsan gum, carrageenan, guar gum, agar, gum arabic, gum ghatti, karaya gum, gum tragacanth, tamarind gum, locust bean gum, and any mixtures thereof.

The embodiments also encompass a method for the production of a bacterial cellulose-containing formulation including the following steps: a) providing a bacterial cellulose product; b) mixing the bacterial cellulose product with at least one precipitation agent; c) co-lysing the mixture of step “b” to remove bacterial cells therefrom; and d) co-precipitating the mixture of step “c” with a water-miscible nonaqueous liquid. In this method, the precipitation agent is selected from the group consisting of a xanthan product, pectin, alginates, gellan gum, welan gum, diutan gum, rhamsan gum, carrageenan, guar gum, agar, gum arabic, gum ghatti, karaya gum, gum tragacanth, tamarind gum, locust bean gum, and any mixtures thereof.

The embodiments described herein further encompass a bacterial cellulose-containing formulation, such as the one produced by the methods described herein. In accordance with one embodiment, the bacterial cellulose-containing formulation includes at least one bacterial cellulose material and at least one polymeric thickener selected from the group consisting of at least one polymer, at least one precipitation agent, and any mixtures thereof.

### DETAILED DESCRIPTION OF THE EMBODIMENTS

The following description is intended to convey a thorough understanding of the embodiments by providing a number of specific embodiments and examples involving a microfibrous cellulose formulation. It is understood, however, that the invention is not limited to these specific embodiments and details, which are exemplary only. It is further understood that one possessing ordinary skill in the art, in light of known formulations, systems and methods, would appreciate the use of the invention for its intended purposes and benefits in any number of alternative embodiments.

It has been discovered that microfibrous cellulose, when formulated with gellan gum and guar gum, with gellan gum and xanthan gum, carrageenan and guar gum, or carrageenan and xanthan gum, is capable of achieving a functionality that is comparable to formulations containing CMC. As a result, new applications, especially food applications, may now be pursued with these novel formulations because they lack CMC, or similar chemical component. Such new applications include, but are not limited to, beverages (including acidified milk drinks), dressings, soups, puddings, etc. Other applications are apparent to one of ordinary skill in the art.

As used herein, the phrase “bacterial cellulose-containing formulation” is intended to encompass a bacterial cellulose product as produced by the inventive method and thus including xanthan product, or other acceptable agents, coating at least of the portion of the resultant bacterial cellulose fiber bundles. The term “formulation,” as used herein, is intended to convey that the product made therefrom is a combination of bacterial cellulose and xanthan, among other agents, produced in such a manner and exhibiting such a resultant structure and configuration. As used herein, the phrase “bacterial cellulose” is intended to encompass any type of cellulose produced via fermentation of a bacteria of the genus Acetobacter and includes materials referred to popularly as microfibrillated cellulose, reticulated bacterial cellulose, and the like.

According to exemplary embodiments, a method for the production of a bacterial cellulose-containing formulation may include the steps of: (a) providing a bacterial cellulose product; (b) mixing bacterial cellulose with a thickener or precipitation agent; (c) lysing the bacterial cells from the bacterial cellulose product or the mixture of the bacterial cellulose and thickener or precipitation agent; and (d) co-precipitating the resultant mixture with a water-miscible non-aqueous liquid.

As noted above, bacterial cellulose may be used as an effective Theological modifier in various compositions. Such materials, when dispersed in fluids, may produce highly viscous, thixotropic mixtures possessing high yield stress. Yield stress is a measure of the force required to initiate flow in a liquid system. Yield stress is indicative of the suspension ability of a fluid, as well as indicative of the ability of the fluid to remain in situ after application to a vertical surface.

Typically, such rheological modification behavior may be provided through some degree of processing of a mixture of the bacterial cellulose in a hydrophilic solvent, such as water, polyols (e.g., ethylene glycol, glycerin, polyethylene glycol, etc.), or mixtures thereof. Such processing is called “activation” and comprises, generally, high pressure homogenization and/or high shear mixing. It has been found that bacterial cellulose-containing formulations of the exemplary embodiments, also will activate with low energy mixing.

During activation the 3-dimensional structure of the cellulose may be modified such that the cellulose can impart functionality to the base solvent or solvent mixture in which the activation occurs, or to a composition to which the activated cellulose is added. As used herein, the term “functionality” includes such properties as thickening, imparting yield stress, heat stability, suspension properties, freeze-thaw stability, flow control, foam stabilization, coating and film formation, and the like. The processing that may be followed during the activation process does significantly more than to just disperse the cellulose in base solvent. Such processing may “tease apart” the cellulose fibers to expand the cellulose fibers.

In various exemplary embodiments, the bacterial cellulose-containing formulation may be provided in the form of a wet slurry (dispersion). In other embodiments, the bacterial cellulose containing formulation may be provided as a dried product, such as one produced by drying the dispersion using well-known drying techniques, such as spray-drying, drum drying or freeze-drying. The activation of the bacterial cellulose (such as MFC or reticulated bacterial cellulose) may expand the cellulose portion to create a reticulated network of highly intermeshed fibers with a very high surface area. For example, activated reticulated bacterial cellulose may possess an extremely high surface area that is thought to be at least 200-fold higher than conventional microcrystalline cellulose (i.e., cellulose provided by plant sources).

In exemplary embodiments, the bacterial cellulose may be of any type associated with the fermentation product of Acetobacter genus microorganisms. For example, see U.S. Patent Application Publication Number 2007/0197779, which is incorporated herein by reference in its entirety. Such aerobic cultured products generally are characterized by a highly reticulated, branching interconnected network of fibers that are insoluble in water.

The preparation of such bacterial cellulose products are commonly known. For example, U.S. Pat. No. 5,079,162 and U.S. Pat. No. 5,144,021, both of which are incorporated by reference herein in their entirety, disclose methods and media for producing reticulated bacterial cellulose aerobically, under agitated culture conditions, using a bacterial strain of Acetobacter aceti var. xylinum. Use of agitated culture conditions may result in sustained production, over an average of 70 hours, of at least 0.1 g/liter per hour of the desired cellulose. Wet cake reticulated cellulose, containing approximately 80-85% water, may be produced using the methods and conditions disclosed in the above-mentioned patent references. Dry reticulated bacterial cellulose may be produced using drying techniques, such as spray-drying, drum drying or freeze-drying, that are well known.

Acetobacter is characteristically a gram-negative, rod shaped bacterium 0.6-0.8 microns by 1.0-4 microns. It is a strictly aerobic organism; that is, metabolism is respiratory, not fermentative. This bacterium may be further characterized by its ability to produce multiple poly β-1,4-glucan chains, which are substantially chemically identical to cellulose. The microcellulose chains, or microfibrils, of reticulated bacterial cellulose may be synthesized at the bacterial surface, at sites external to the cell membrane. These microfibrils may generally have cross sectional dimensions of about 1.6 nm by 5.8 nm. In contrast, under static or standing culture conditions, the microfibrils at the bacterial surface combine to form a fibril generally having cross sectional dimensions of about 3.2 nm by 133 nm. It is believed that the small cross sectional size of these Acetobacter-produced fibrils, together with the concomitantly large surface and the inherent hydrophilicity of cellulose, may provide a cellulose product having an unusually high capacity for absorbing aqueous solutions. Additives may be used in combination with the reticulated bacterial cellulose to aid in the formation of stable, viscous dispersions.

The aforementioned problems believed to be inherent in the purification and collection of such bacterial cellulose have led to the method described in U.S. Patent Application Publication Number 2007/0197779. As described, the first step in the overall process is to provide the target bacterial cellulose in fermented form.

In the exemplary embodiments, the bacterial cellulose product may be purified, such as by lysing. Purification is well known for such materials. Lysing of the bacterial cells from the bacterial cellulose product may be accomplished through the introduction of a caustic, such as sodium hydroxide, or any additive having like high pH (e.g., above about 12.5), in an amount sufficient to properly remove as many expired bacterial cells as possible from the cellulosic product. This may be performed in more than one step if desired. Typically, this is followed by neutralization with an acid. Any suitable acid of sufficiently low pH and molarity may be utilized in this step provided that the acid may effectively neutralize or reduce the pH level of the product as close to 7.0 as possible. Exemplary neutralizing agents include, for example, sulfuric acid, hydrochloric, and nitric acid. One of ordinary skill in the art could easily select a suitable neutralizing agent and specify an appropriate amount of such a reactant for such a purpose, using the guidance provided herein.

In exemplary embodiments, the cells may be lysed and digested through enzymatic methods, such as, for example, treatment with lysozyme and protease at the appropriate pH. Suitable methods are understood by those having ordinary skill in the art.

In exemplary embodiments, the lysed product may be subjected to mixing with a polymeric thickener and/or precipitation agent in order to effectively coat the target fibers and bundles of the bacterial cellulose. In exemplary embodiments, the polymeric thickener should be insoluble in alcohol (in particular, isopropyl alcohol). Such a thickener may be either an aid for dispersion of the bacterial cellulose within a target fluid composition, or an aid in drying the bacterial cellulose to remove water therefrom more easily, as well as potentially an aid in dispersing or suspending the fibers within a target fluid composition. Suitable dispersing aids (agents) include, without limitation, cationic guar, cationic hydroxyethyl cellulose (HEC), etc.—in essence any compound that is polymeric in nature and exhibits the necessary dispersion capabilities for the bacterial cellulose fibers when introduced within a target liquid solution. Suitable precipitation aids (agents), as noted above, include any number of biogums, including xanthan products (such as KELTROL®, KELTROL T®, and the like from CP Kelco), gellan gum, welan gum, diutan gum, rhamsan gum, and the like, and other types of natural polymeric thickeners, such as pectin, guar, locust bean gum, as a few non-limiting examples. In certain exemplary embodiments, the polymeric thickener is a xanthan product and is introduced and mixed with the bacterial cellulose in a broth form. It is believed that the coming of the two products in broth, powder or rehydrated powder form, enables the desired generation of a xanthan coating on at least a portion of the fibers and/or bundles of the bacterial cellulose. In one embodiment, the broths of bacterial cellulose and xanthan are mixed subsequent to purification (lysing) of both in order to remove the residual bacterial cells. In another embodiment, the broths may be mixed together without lysing initially, but co-lysed during mixing for such purification to occur.

In various exemplary embodiments, the bacterial cellulose may be present in an amount from about 0.1% to about 5% by weight in the mixture, and more preferably from about 0.5 to about 3.0%. In various exemplary embodiments, the polymeric thickener may be present in an amount from about 0.1 to about 10% by weight in the mixture.

In the exemplary embodiments, after mixing and coating of the bacterial cellulose by the polymeric thickener, the resultant product may be collected through co-precipitation in a water-miscible nonaqueous liquid. In certain exemplary embodiments, for toxicity, availability, and cost reasons, such a liquid is an alcohol, such as, for example, isopropyl alcohol. Other suitable alcohols include, for example, ethanol, methanol, butanol, and the like. Other exemplary water-miscible nonaqeuous liquids include, for example, acetone, ethyl acetate, and the like. Any mixtures of the foregoing nonaqueous liquids also may be utilized for a co-precipitation step. In exemplary embodiments, the co-precipitated product may be processed through a solid-liquid separation apparatus, allowing for the alcohol-soluble components to be removed, leaving the desired bacterial cellulose-containing formulation thereon.

In exemplary embodiments, a press cake form product may be collected from the co-precipitation step. In various exemplary embodiments, this press cake form product may be transferred to a drying apparatus, and subsequently milled to produce a dried product with a predetermined particle size. Further co-agents may be added to the press cake or to the dried materials in order to provide further properties and/or benefits Such exemplary co-agents include plant, algal and bacterial polysaccharides and their derivatives along with lower molecular weight carbohydrates such as sucrose, glucose, maltodextrin, and the like.

In exemplary embodiments, the bacterial cellulose-containing formulations prepared by the exemplary methods described herein, may include at least one bacterial cellulose material and at least one polymeric thickener. Depending on the method used, the polymeric thickener may be a polymer, a precipitation agent, or a combination thereof. The exemplary bacterial cellulose-containing formulation also may include one or more other additives that have been used in a method of preparation. Other additives that may be present within the bacterial cellulose-containing formulation include, without limitation, a hydrocolloid, starch (and like sugar-based molecules), modified starch, animal-derived gelatin, and non-charged cellulose ethers (such as carboxymethylcellulose, hydroxyethylcellulose, and the like).

In exemplary embodiments, the bacterial cellulose-containing formulations produced by the methods described herein, may be introduced into a plethora of possible food compositions, including, for example: beverages, frozen products, cultured dairy, and the like; non-food compositions, such as household cleaners, fabric conditioners, hair conditioners, hair styling products, or as stabilizers or formulating agents for asphalt emulsions, pesticides, corrosion inhibitors in metal working, latex manufacture, as well as in paper and non-woven applications, biomedical applications, pharmaceutical excipients, and oil drilling fluids, etc. Exemplary fluid compositions, prepared as described above, may include such bacterial cellulose-containing formulations in an amount from about 0.01% to about 1% by weight, and preferably about 0.03% to about 0.5% by weight of the total weight of the fluid composition. The bacterial cellulose-containing formulation of the exemplary embodiments should impart a viscosity modification to water sample of 500 mL (when added in an amount of at most 0.30% by weight thereof) of at least 10 cps as well as a yield stress measurement within the same test sample of at least 0.1 dynes/cm².

### EXAMPLES

The following non-limiting examples provide teachings of various methods and formulations that are encompassed within the exemplary embodiments.

### Example 1

MFC broth was produced in a 1200 gallon fermentor with final yield of 1.51 wt %. The broth was treated with 350 ppm of hypo chlorite. It was then treated with 70 ppm of lysozyme and 194 ppm of protease. A portion of the treated MFC broth was mixed with given amount of guar and cationic guar solutions (ratio MFC/Guar/Cationic Guar=5/2/3, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 58.6 cP.

### Example 2

MFC broth was produced in a 1200 gal fermentor with final yield of 1.51 wt %. The broth was treated with 350 ppm of hypo chlorite. It was then treated with 70 ppm of lysozyme and 194 ppm of protease. A portion of the treated MFC broth was mixed with given amount of cationic guar solution (ratio MFC/Cationic Guar=6/4, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 83.4 cP.

### Example 3

MFC broth was produced in a 1200 gal fermentor with final yield of 1.51 wt %. The broth was treated with 350 ppm of hypo chlorite. It was then treated with 70 ppm of lysozyme and 194 ppm of protease. A portion of the treated MFC broth was mixed with given amount of cationic starch solution (ratio MFC/Cationic Starch=1/1, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 23.0 cP.

### Example 4

MFC broth was produced in a 1200 gal fermentor with final yield of 1.55 wt %. The broth was treated with 350 ppm of hypo chlorite. It was then treated with 70 ppm of lysozyme and 194 ppm of protease. A portion of the treated MFC broth was mixed with given amount of Kelcogel Gellan and guar solutions (ratio MFC/Kelcogel Gellan/Guar=5/3/2, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 65.2 cP.

### Example 5

MFC broth was produced in a 1200 gal fermentor with final yield of 1.55 wt %. The broth was treated with 350 ppm of hypo chlorite. It was then treated with 70 ppm of lysozyme and 194 ppm of protease. A portion of the treated MFC broth was mixed with given amount of carrageenan and guar solutions (ratio MFC/Ca/Tageenan/Guar=5/3/2, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 75.0 cP.

### Example 6

MFC broth was produced in a 1200 gal fermentor with final yield of 1.55 wt %. The broth was treated with 350 ppm of hypo chlorite. It was then treated with 70 ppm of lysozyme and 194 ppm of protease. A portion of the treated MFC broth was mixed with given amount of Kelcogel Gellan and guar solutions (ratio MFC/Kelcogel Gellan/Guar=3/1/1, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 37.5 cP.

### Example 7

MFC broth was produced in a 1200 gal fermentor with final yield of 1.55 wt %. The broth was treated with 350 ppm of hypo chlorite. It was then treated with 70 ppm of lysozyme and 194 ppm of protease. A portion of the treated MFC broth was mixed with given amount of Kelcogel Gellan and guar solutions (ratio MFC/Kelcogel Gellan/Guar=6/1/3, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 33.6 cP.

### Example 8

MFC broth was produced in a 1200 gal fermentor with final yield of 1.55 wt %. The broth was treated with 350 ppm of hypo chlorite. It was then treated with 70 ppm of lysozyme and 194 ppm of protease. A portion of the treated MFC broth was mixed with given amount of Kelcogel Gellan and guar solutions (ratio MFC/Kelcogel Gellan/Guar=6/3/1, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 23.3 cP.

### Example 9

MFC broth was produced in a 1200 gal fermentor with final yield of 1.55 wt %. The broth was treated with 350 ppm of hypo chlorite. It was then treated with 70 ppm of lysozyme and 194 ppm of protease. A portion of the treated MFC broth was mixed with given amount of carrageenan and guar solutions (ratio MFC/Carrageenan/Guar=3/1/1, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 56.5 cP.

### Example 10

MFC broth was produced in a 1200 gal fermentor with final yield of 1.55 wt %. The broth was treated with 350 ppm of hypo chlorite. It was then treated with 70 ppm of lysozyme and 194 ppm of protease. A portion of the treated MFC broth was mixed with given amount of carrageenan and guar solutions (ratio MFC/Carrageenan/Guar=6/1/3, dry basis) at bench. The mixture was then precipitated with IPA (85%). The press cake was dried and milled at bench. After activation with an APV homogenizer at 2500 psi, the product viscosity (0.3 wt/wt %) measured by a Brookfield viscometer at 60 rpm (Spindle 61), in standard tap water (STW) was 35.2 cP.

### Example 11

A simplified anti-bacterial hard surface cleaner containing 4% benzylalkonium chloride with suspended alginate beads was prepared. The cleaner exhibited a measurable yield value and possessed the ability to suspend air bubbles and beads. A yield value of 0.82 Pa (as measured with a Brookfield® Yield Rheometer) was obtained. A concentrate was first prepared containing 0.3% microfibrous cellulose blend (MFC/cationic guar 1:1 blend) in deionized water. The concentrate was made by mixing the solution on an Oster® blender at “liquefy” (top speed) for 5 minutes. The microfibrous cellulose mixture was then diluted 1:1 with an 8% solution of benzylalkonium chloride. The cationic solution was added to the microfibrous cellulose solution while mixing at about 600 rpm with a jiffy mixing blade. Alginate beads were added to demonstrate suspension. Excellent suspension of air and/or alginate beads was achieved with no settling observed at room temperature or at 45° C. for 3 months. The microfibrous cellulose diluted well notwithstanding the relative low shear of the jiffy or propeller mixing blade.

### Example 12

A concentrated commercial fabric softener containing about 7.5% cationic surfactant was prepared. “Downy® Clean Breeze™ Ultra Concentrated” liquid fabric softener was modified with MFC. A 0.3% microfibrous cellulose blend (MFC/cationic guar 1:1 blend) concentrate was activated in distilled water with an Oster® blender set at top speed (liquefy) by mixing for 5 minutes. The microfibrous cellulose solution was diluted 1:1 with Downy® ultra concentrated fabric softener while mixing at about 600 rpm with a jiffy mixing blade. Alginate beads were added to test suspension. Very good suspension of the beads was achieved for the dilution resulting in a yield point of 1.4 Pa (as measured with a Brookfield® Yield Rheometer). The fabric softener was put in a 45° C. oven to assess heat stability and showed excellent stability with no loss in suspension over 4 weeks of aging.

### Example 13

A conditioning hair spray with glitter suspended therein was prepared. The resulting hair spray exhibited good spray characteristics and excellent suspension properties. A yield value of about 0.2 Pa (as measured with a Brookfield® Yield Rheometer) was obtained. The hair spray was prepared using the following method, and recipe (summarized in Table 1):

Step A: Deionizied water and disodium EDTA were added to a small Oster® mixing jar. Microfibrous cellulose (MFC/cationic guar 6:4 blend) was added to the top of the water and then the Oster® mixer blade was assembled and the combination was mixed at top speed for 5 minutes (“Liquify” speed).

Step B: STS and fragrance were mixed with pre-warmed RH-40 and propylene glycol and solubilized in the water phase.

Step C: The remaining ingredients were added sequentially and mixed. The result was a low viscosity, sprayable hair conditioner with glitter suspended therein and a pH of 4.8.

TABLE 1
Sprayable Hair Conditioner with Suspension Properties

| Process Step | Ingredient | % (w/w) | Grams |
|---|---|---|---|
| A | Deionized Water | 93.725 | 374.9 |
| A | Microfibrous Cellulose blend (MFC/ | 0.125 | 0.5 |
| | cationic guar 6:4 blend) | | |
| A | Disodium EDTA | 0.1 | 0.4 |
| B | Fragrance | To Suit | |
| B | Crodamol STS | 0.5 | 2 |
| B | Cremophor RH 40 | 1.5 | 6 |
| B | Propylene glycol | 0.75 | 3 |
| C | CTAC 29 | 1 | 4 |
| | (29% Cetrimonium Chloride, | | |
| | a cationic conditioning agent) | | |
| C | Wheat Protein | 1 | 4 |
| C | Panthenol | 0.2 | 0.8 |
| C | Acetamide MEA | 1 | 4 |
| C | Kathon | 0.1 | 0.4 |
| C | Color | To Suit | To Suit |
| C | Glitter | To Suit | To Suit |
| Totals | | 100.00 | 400.00 |

Each sample exhibited excellent and highly desirable viscosity modification and yield stress results. In terms of bacterial cellulose products, such results have been heretofore unattainable with the low complexity methods followed herein.

While the invention is described and disclosed in connection with certain exemplary embodiments and practices, it is in no way intended to limit the invention to those specific embodiments, rather it is intended to cover equivalent structures and all alternative embodiments and modifications as may be defined by the scope of the appended claims and equivalence thereto.

What is claimed:

1. A method for the production of a bacterial cellulose-containing formulation comprising the steps of
a) providing a bacterial cellulose product;
b) optionally lysing the bacterial cells from the bacterial cellulose product;
c) mixing said bacterial cellulose product of either step “a” or step “b” with a charged polymeric thickener selected from the group consisting of at least one polymer, at least one precipitation agent, and any combination thereof; and
d) co-precipitating the mixture of step “c” with a water-miscible non-aqueous liquid.

2. The method of claim 1 wherein said polymeric thickener of step “c” is selected from the group consisting of xanthan gum, pectin, alginates, gellan gum, welan gum, diutan gum, rhamsan gum, carrageenan, guar gum, agar, gum arabic, gum ghatti, karaya gum, gum tragacanth, tamarind gum, locust bean gum, and any mixtures thereof.

3. The method of claim 2 wherein said precipitation agent is an alcohol.

4. The method of claim 1 wherein said polymeric thickener of step “c” is a cationic guar or cationic hydroxyethyl cellulose.

5. The method of claim 1 wherein said polymeric thickener of step “c” is a precipitation agent.

6. The method of claim 5 wherein said precipitation agent is selected from the group consisting of a xanthan product, pectin, alginates, gellan gum, welan gum, diutan gum, rhamsan gum, carrageenan, guar gum, agar, gum arabic, gum ghatti, karaya gum, gum tragacanth, tamarind gum, locust bean gum, and any mixtures thereof.

7. The method of claim 1 wherein said bacterial cellulose product is a microfibrillated cellulose.

8. The method of claim 7 wherein said polymeric thickener of step “c” is selected from the group consisting of guar gum, gellan gum, carrageenan, and any mixtures thereof.

9. The method of claim 7 wherein said polymeric thickener of step “c” is a precipitation agent.

10. The method of claim 9 wherein said precipitation agent is selected from the group consisting of a xanthan product, pectin, alginates, gellan gum, diutan gum, welan gum, rhamsan gum, carrageenan, guar gum, agar, gum arabic, gum ghatti, karaya gum, gum tragacanth, tamarind gum, locust bean gum, and any mixtures thereof.

11. The method of claim 10 wherein said precipitation agent is selected from the group consisting of xanthan, pectin, diutan gum, and any mixtures thereof.

12. A method for the production of a bacterial cellulose-containing formulation comprising the steps of
a) providing a bacterial cellulose product;
b) optionally lysing the bacterial cells from the bacterial cellulose product;
c) mixing said resulting bacterial cellulose product of either step “a” or step “b” with at least one precipitation agent selected from the group consisting of a xanthan product, pectin, alginates, gellan gum, welan gum, diutan gum, rhamsan gum, carrageenan, guar gum, agar, gum arabic, gum ghatti, karaya gum, gum tragacanth, tamarind gum, locust bean gum, and any mixtures thereof; and
d) co-precipitating the mixture of step “c” with a water-miscible non-aqueous liquid.

13. The method of claim 12 wherein said precipitation agent is selected from the group consisting of xanthan, pectin, diutan gum, and any mixtures thereof.

14. A method for the production of a bacterial cellulose-containing formulation comprising the steps of
a) providing a bacterial cellulose product;
b) mixing said bacterial cellulose product with at least one precipitation agent selected from the group consisting of a xanthan product, pectin, alginates, gellan gum, welan gum, diutan gum, rhamsan gum, carrageenan, guar gum, agar, gum arabic, gum ghatti, karaya gum, gum tragacanth, tamarind gum, locust bean gum, and any mixtures thereof;
c) co-lysing the mixture of step “b” to remove bacterial cells therefrom; and
d) co-precipitating the mixture of step “c” with a water-miscible non-aqueous liquid.

15. The method of claim 14 wherein said precipitation agent is selected from the group consisting of xanthan, pectin, diutan gum, and any mixtures thereof.

16. A bacterial cellulose-containing formulation comprising at least one bacterial cellulose material and at least one polymeric thickener selected from the group consisting of at least one polymer, at least one precipitation agent, and any mixtures thereof.

17. The formulation of claim 16 wherein said polymeric thickener is selected from the group consisting of xanthan gum, pectin, alginates, gellan gum, welan gum, diutan gum, rhamsan gum, carrageenan, guar gum, agar, gum arabic, gum ghatti, karaya gum, gum tragacanth, tamarind gum, locust bean gum, and any mixtures thereof

18. The formulation of claim 16 wherein said polymeric thickener is a precipitation agent.

19. The formulation of claim 18 wherein said precipitation agent is selected from the group consisting of a xanthan gum, pectin, alginates, gellan gum, welan gum, rhamsan gum, carrageenan, guar gum, agar, gum arabic, gum ghatti, karaya gum, gum tragacanth, tamarind gum, locust bean gum, and any mixtures thereof.

20. The formulation of claim 16 wherein said bacterial cellulose product is microfibrillated cellulose.

21. The formulation of claim 20 wherein said precipitation agent is selected from the group consisting of xanthan, pectin, diutan gum, and any mixtures thereof.

22. The formulation of claim 21 wherein said precipitation agent is xanthan.

23. The formulation of claim 21 wherein said precipitation agent is pectin.

24. The formulation of claim 21 wherein said precipitation agent is diutan gum.
